(19) United States
(12) Patent Application Publication
(10) Pub. No.: US 2009/0105644 A1
(43) Pub. Date: Apr. 23, 2009

(54) INTRAVASCULAR MEDICAL DEVICE HAVING A READILY COLLAPSIBLE COVERED FRAME

(75) Inventors: Michael J. Leonard, Olympia, CA (US); William E. Webler, Escondido, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS INC., Santa Clara, CA (US)

(21) Appl. No.: 11/876,555

(22) Filed: Oct. 22, 2007

(51) Int. Cl. A61M 29/00; A61M 25/00

(52) U.S. Cl. 604/104; 604/523

(57) ABSTRACT

An elongated intravascular device having a frame configured for reversibly expanding in a patient's body lumen, which has a sleeve secured to the frame, and at least one sleeve-folding strut configured to fold the sleeve inwardly as the frame radially collapses in the patient's body lumen. Additional aspects of the invention are directed to methods of recovering such expanded frame type devices, and a recovery catheter configured for collapsing an expanded frame. The devices and methods of the invention facilitate the collapse of expanded frame devices, for repositioning or removal from the patient's body lumen.

### CROSS-REFERENCES TO RELATED APPLICATIONS

None

### BACKGROUND OF THE INVENTION

The present invention relates to intravascular medical devices, and more particularly to an elongated catheter or wire for use in an interventional procedure in a patient's blood vessel.

A variety of non-surgical interventional procedures have been developed over the years for opening stenosed or occluded blood vessels in a patient caused by the build up of plaque or other substances on the walls of the blood vessel. Such procedures usually involve the percutaneous, transluminal introduction into the occluded vessel of an interventional device configured to treat the occlusion by one or more commonly known methods including dilatation, stent implantation, atherectomy, and drug delivery. For example, in PTCA, a balloon catheter is inserted into the patient's arterial system and is advanced and manipulated to position the catheter balloon along the stenosed region in the artery, and the balloon is inflated to compress the plaque to thereby open the occluded region. The balloon is then deflated and the balloon catheter removed from the blood vessel.

In such procedures, interventional devices are generally known which have an operative distal end with a reversibly expandable frame, for example for use as a temporary stent or embolic protection device. When used for embolic protection, the frame is typically secured to a membrane, to form a filter or trap which is positioned in the blood vessel downstream from the treatment site and radially expanded to capture embolic debris released during the interventional procedure, and then collapsed at the end of the procedure for removal from the patient. A variety of design structures have been suggested to enable the reversible expansion and collapse of such frame structures including frames which are self-expanding, or which expand and/or collapse by activation of a pull or push wire or other mechanism, and/or which collapse upon being slid into a recovery catheter. Complications encountered during collapse of the device in the body lumen will lengthen the duration of the procedure and can be potentially harmful to the patient, if for example the membrane on the frame tears or dislodges during collapse of the frame.

What has been needed is an interventional device having a reversibly expandable frame that can be rapidly and safely collapsed within the body lumen for removal or repositioning of the device. This invention satisfies these and other needs.

### SUMMARY OF THE INVENTION

The invention is directed to an elongated intravascular device having a frame configured for reversibly expanding in a patient's body lumen, which has a sleeve secured to the frame, and at least one sleeve-folding strut configured to fold the sleeve inwardly as the frame radially collapses in the patient's body lumen. Additional aspects of the invention are directed to methods of recovering such expanded frame type devices, and a recovery catheter configured for collapsing an expanded frame. The devices and methods of the invention facilitate the collapse of expanded frame devices, for repositioning or removal from the patient's body lumen.

The device generally comprises an elongated shaft with a distal shaft section, and a frame on the distal shaft section which is configured to transform from a low profile collapsed configuration to a radially expanded configuration in the patient's body lumen, and then to radially collapse from the expanded configuration as a recovery catheter is slidably advanced over the expanded frame. The frame is formed in part by a plurality of struts, and has a proximal end, a distal end, at least one or more typically at least three sleeve-folding strut(s), and a sleeve fixedly secured to the struts. The sleeve has an open first end forming a sleeve mouth located between the proximal and distal ends of the frame, and an opposite end, such that the frame has a first longitudinal section along which the sleeve does not extend and a second longitudinal section along which the sleeve does extend. The sleeve-folding strut(s) extend at least along at least a part of the first (sleeve-free) longitudinal section of the frame, and have a larger outer diameter in the expanded configuration than strut portions circumferentially adjacent thereto, to radially collapse prior to the circumferentially adjacent portions and thereby fold the sleeve inwardly as the frame radially collapses.

A method of recovering a device having an elongated shaft and an expanded frame on a distal shaft section configured for reversibly transforming from a radially expanded configuration to a collapsed configuration in a patient's body lumen, generally comprises slidably advancing a recovery catheter over the device to position a distal end of the recovery catheter proximally adjacent to the expanded frame, and radially collapsing the frame by slidably disposing the frame within the recovery catheter as the sleeve is forced to fold inwardly and prevented or inhibited from bunching or folding outwardly as the frame radially collapses. Preferably, the frame comprises the plurality of struts and at least one sleeve folding strut as discussed above, such that by slidably disposing the first longitudinal section of the frame within the recovery catheter, the sleeve-folding strut contacts and is radially collapsed by the recovery catheter prior to the circumferentially adjacent strut portions, to thereby fold the sleeve inwardly as the frame radially collapses.

In a presently preferred embodiment, the device is a catheter configured for infusing an agent into the patient's body lumen, such that the elongated device shaft is a tubular member having at least one lumen therein extending from the proximal end of the shaft to a fluid delivery port in the distal shaft section. The catheter is preferably a drug delivery catheter used for infusing a therapeutic agent into the patient's body lumen, and in a presently preferred embodiment, the agent is an anti-inflammatory agent (e.g. steroids), or is an agent that induces cholesterol efflux from arterial wall plaque (e.g. ApoA1 mimetic peptides, PPARα agonists). However, a variety of suitable agents can be delivered using the catheter of the invention including diagnostic agents, perfusion agents (e.g., oxygenated fluid or blood), or merely a flushing agent (e.g., saline or contrast).

In a presently preferred embodiment, the sleeve is a solid-walled member configured to occlude the patient's body lumen when the frame is in the expanded configuration. In the embodiment having an agent infusion lumen in the device shaft, the occluding frame provides for improved delivery of the agent within the blood vessel by reducing the flow of blood along the agent delivery port to thereby increase the residence time of the agent at the treatment location within the blood vessel by reducing agent wash-out in the blood vessel.

One aspect of the invention is directed to a recovery catheter having an elongated shaft with a porous wall, and a lumen therein dimensioned for slidably advancing over a device which has a reversibly expandable frame to thereby collapse the expanded frame to a collapsed configuration. The recovery catheter of the invention generally comprises an elongated shaft having a porous wall at least along a distal recovery section of the shaft, with a porosity configured to allow fluid forced by pressurization through the porous wall as the frame is collapsed into the recovery section. The porosity is sufficiently small such that the porous wall has a sufficiently high column strength for collapsing the frame. As a result, fluid (e.g., blood and contrast) which otherwise would have been trapped in and around the sleeved frame as it is collapsing is allowed to escape via the porous section of the recovery catheter. Fluid pressure, which otherwise would build and inhibit recovery of the frame as an occluding frame collapses, is thus released when the fluid flows out the porous wall of the recovery catheter. The porous wall avoids the need to aspirate the trapped fluid by vacuum force at the proximal end of the recovery catheter. In one embodiment, the porous wall recovery catheter is part of a catheter system, configured to slidably advance over the device having the reversibly collapsible frame with a sleeve-folding strut.

The devices and methods of the invention facilitate the collapse of expanded frame devices, for repositioning or removal from the patient's body lumen. The devices of the invention are particularly useful in providing the ability to quickly, easily, and safely collapse, reposition and then re-expand the frame repeatedly in the patient's body lumen. Specifically, by providing the sleeve-folding strut(s), the sleeved frame will collapse without the sleeve material bunching or folding outside of the frame in a way which would have inhibited recovery of the device by engaging with the recovery catheter. As a recovery catheter slips over the collapsing frame, such bunched sleeve material can lock-up the device within the recovery catheter lumen, and forcing the device into the recovery catheter lumen can cause the sleeve to be damaged or torn off the frame. Moreover, the devices are preferably highly maneuverable, to facilitate positioning the device distal end at a desired location within the body lumen. Additionally, a porous recovery catheter of the invention has a porosity sufficient to allow for ample fluid pressure release but without affecting the structural integrity of the recovery catheter. These and other advantages of the invention will become more apparent from the following detailed description of the invention and accompanying exemplary drawings.

### BRIEF DESCRIPTION OF THE DRAWINGS

FIG. 1 is an elevational, partially in section, view of a device embodying features of the invention, having a reversibly expandable frame.

FIGS. 2-6 are transverse cross sectional views of the device of FIG. 1, taken along lines 2-2, 3-3, 4A-4A, 4B-4B, 5-5, and 6-6, respectively.

FIG. 7 illustrates an enlarged longitudinal cross section of the covered expanded frame of the device of FIG. 1.

FIGS. 8-10 illustrate a device assembly embodying features of the invention during collapse of the frame of the device into a recovery catheter, with FIG. 8 showing the device with the recovery catheter advanced to a location proximally adjacent to the frame.

FIG. 9 illustrates the device assembly of FIG. 8 with the recovery catheter advanced distally over the frame to partially collapse the frame.

FIG. 9A illustrates a transverse cross section of the device assembly of FIG. 9, taken along line 9A-9A.

FIG. 10 illustrates the device assembly of FIG. 9 with the recovery catheter advanced further distally over the frame to fully collapse the frame.

FIG. 10A illustrates a transverse cross section of the device assembly of FIG. 10, taken along line 10A-10A.

FIG. 11 is an elevational view, partially in section, of a porous recovery catheter embodying features of the invention.

FIG. 12 illustrates a transverse cross sectional view of the porous recovery catheter of FIG. 11, taken along line 12-12.

### DETAILED DESCRIPTION OF THE PREFERRED EMBODIMENTS

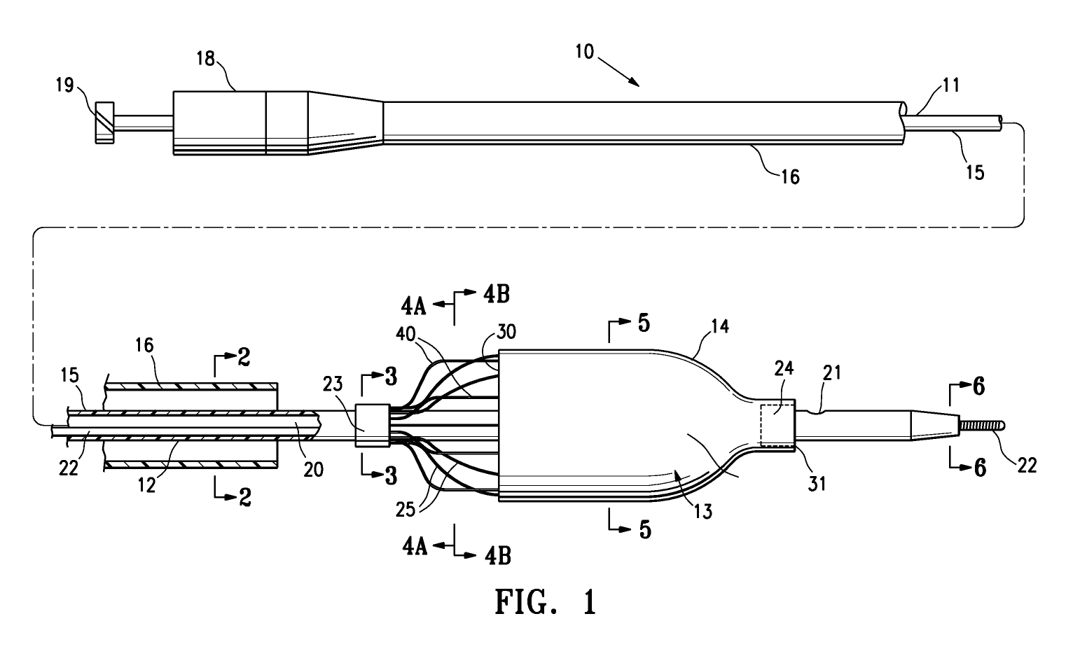

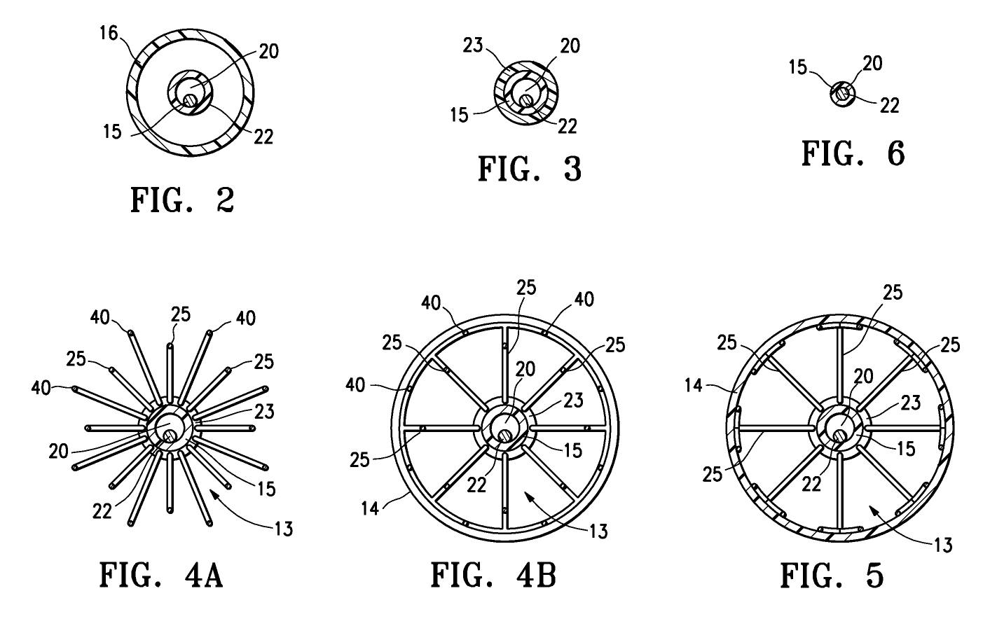

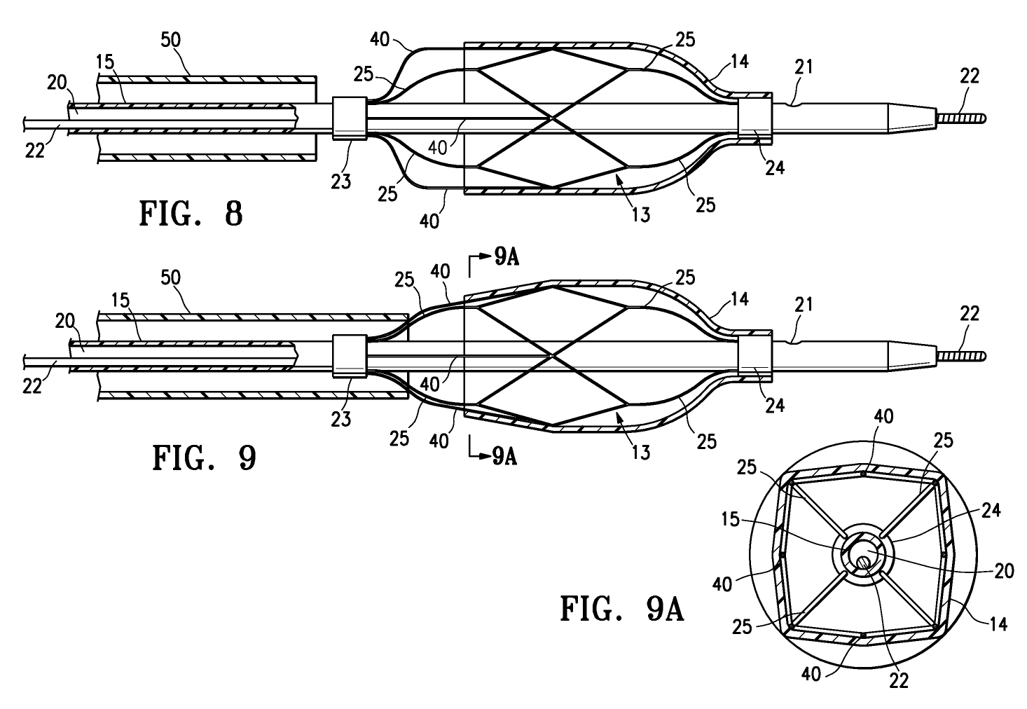

FIG. 1 illustrates an elevational view, partially in section, of an intravascular catheter 10 embodying features of the invention, generally comprising an elongated shaft 11 having a distal shaft section 12 with an expandable frame 13 configured to radially expand to an expanded configuration in a patient's body lumen, and then radially collapse from the expanded configuration as a recovery catheter 50 (see FIGS. 8-10) is slidably advanced over the frame 13. The frame 13 has a sleeve 14 secured thereto. The catheter 10 is advanced within a patient's body lumen with the frame 13 in a low-profile collapsed configuration, and once positioned at a desired site in the body lumen, the frame is allowed to or caused to open and radially expand to the expanded configuration for performing an interventional procedure. In FIG. 1, the frame is illustrated in the expanded configuration. FIGS. 2-5 illustrate transverse cross sectional views of the device of FIG. 1, taken along lines 2-2, 3-3, 4A-4A, 4B-4B, and 5-5, respectively.

In the illustrated embodiment the shaft 11 comprises an inner tubular member 15, and an outer sheath member 16 slidably disposed on the inner tubular member. The frame 13 is fixedly secured to the inner tubular member 15, and is configured to radially self-expand to an expanded configuration by release of a radially restraining force, which in the illustrated embodiment is provided by the shaft outer member 16. Thus, the frame 13 is biased to automatically radially expand to the expanded configuration by slidably displacing the frame 13 and the outer member 16 relative to one another, such that the frame 13 deploys upon becoming distally spaced from the distal end of the outer member 16. The frame is typically deployed to the expanded configuration by proximally withdrawing the outer member 16 while holding the inner member 15 stationary to maintain the position of the frame within the body lumen 19. Although less preferred, due in part to the potential for damage to the vessel wall, the inner member 15 can alternatively or additionally be advanced distally during deployment of the frame 13. The outer sheath member 16 is typically configured to be peeled or otherwise removed from the inner tubular member 15 during deployment of the frame 13. For example, although not illustrated, the outer sheath member 16 typically has a weakened wall portion extending along the length thereof, so that as the outer sheath member 16 is proximally retracted it is caused to peel off the inner tubular member 15 at the proximal end of the catheter 10. In alternative embodiments, the outer sheath member 16 is not designed to be removed from the inner tubular member 15 during use. A proximal adapter 18 having a port 19 configured for connecting to a fluid agent source (not shown) is on the proximal end of the catheter 10. The adapter can be configured to facilitate displacing the outer member 16 of the shaft 11 relative to the inner member 15 to deploy the frame 13 (primarily in embodiments in which the outer sheath member 16 is not designed to be removed from the inner member, similar to conventional adapters or handles on self-expanding embolic protection filters and stent delivery systems. For recovery, a separate recovery catheter is advanced over the shaft 11 (inner member 15 with outer member 16 thereon, or inner member 15 only after removal of outer member 16) to collapsed the covered frame 13 after a procedure. Alternatively, the outer member 16 is configured to be readvanced over the expanded frame in order to recover the device.

In the illustrated embodiment, the catheter 10 is an agent delivery catheter. The inner tubular member 15 has an agent delivery lumen 20 extending from the proximal end of the shaft to an agent delivery port 21 in the distal shaft section, and the sleeve 14 on the frame 13 is a solid-walled member configured to occlude the body lumen. In the expanded configuration, the sleeve defines an open proximal end 30 and a closed distal end 31, so that the interior of the sleeve acts as a trap which prevents the flow of blood through the wall of the sleeve to thereby decrease the flow of blood along the agent delivery port 21. The agent delivery port 21 is located distal to the distal end of the interior of the occluding sleeve 14, to decrease the blood flow which otherwise would dilute and carry away agent infused from the agent delivery port 21. The agent delivery catheter 10 can be provided with additional or alternative agent delivery ports at a variety of suitable locations along the shaft 11, typically along the distal shaft section, preferably distal to the frame 13. Additionally, although illustrated with a single covered frame 13, the catheter could alternatively have multiple frames longitudinally spaced apart along the shaft.

A wire 22 extends along the length of the inner tubular member 15 from the proximal to the distal end of the device 10, with a floppy distal tip at the distal end of the catheter shaft 11 to facilitate advancing the catheter 10 in the patient's tortuous vasculature. In the illustrated embodiment, the wire 22 is located within the agent delivery lumen 20, although a variety of suitable shaft configurations can alternatively be used which generally provide an agent delivery lumen and an advanceable shaft for supporting the frame 13. In the illustrated embodiment, the wire 22 is a core wire which is fixed to the inner tubular member and which provides the shaft with the general support and pushability required. Distal to the agent delivery port 21, the shaft 11 in the illustrated embodiment closes down onto the fixed core wire 22 (see FIG. 6), such that fluid agent in the lumen 20 is forced to exit the shaft via the port 21. In another embodiment, the wire 22 is a guidewire and the shaft 11 is configured to be slidably advanceable over the guidewire for positioning the catheter 10 in the patient's body lumen. The guidewire can be slidably disposed in a variety of suitable shaft designs, including having the guidewire slidably disposed in the agent delivery lumen 20 within the inner tubular member 15, or having a dedicated guidewire lumen (in addition to an agent delivery lumen) configured to slidably receive the guidewire, in a relatively short rapid exchange guidewire lumen or a full length guidewire lumen.

The frame 13 has a proximal end and a distal end which generally comprise an annular proximal skirt section 23, and an annular distal skirt section 24 (shown in dashed-line under the sleeve 14 in FIG. 1), respectively, for mounting the frame on the inner tubular member 15 in the illustrated embodiment. The expandable portion of the frame 13 is formed by a plurality of struts 25 which extend from the proximal to the distal skirt section 23, 24 of the frame. To allow the frame to expand and collapse, one of the annular skirt sections (typically the proximal skirt section 23) is fixedly mounted and the opposite skirt section (e.g., the distal skirt section 24) is slidably mounted on the inner member 15. Thus, the distal skirt section 24, typically comprising a polymeric or metal ring, will slide distally on the inner member 15 as the frame 13 radially collapses from the expanded configuration shown in FIG. 1. The sliding distal skirt section 24 is typically closely mounted around the inner member 15 so that there is significant resistance to the flow of fluid between the outer surface of the inner member 15 and the inner surface of the sliding distal skirt section. Fixedly (i.e., non-moveably bonding) mounting one of the skirt sections of the frame to the shaft can be achieved using a variety of suitable configurations and methods including adhesively bonding the mating surfaces. Although illustrated as a ring member, the skirt section should be understood to refer to a variety of suitable structural configurations that mount the frame struts on the shaft, including directly bonding the struts thereto.

The frame struts in the expanded configuration form a generally tubular body between conical proximal and distal ends extending down to the skirt sections 23, 24 of the frame in the expanded configuration. From the collapsed configuration, the network of struts 25 articulate to expand the tubular body of the frame radially in all directions (i.e., around the entire circumference of the frame) to the expanded diameter. In addition to the struts 25 (hereinafter “structural struts 25”) the frame has at least one sleeve-folding strut 40 discussed in detail below.

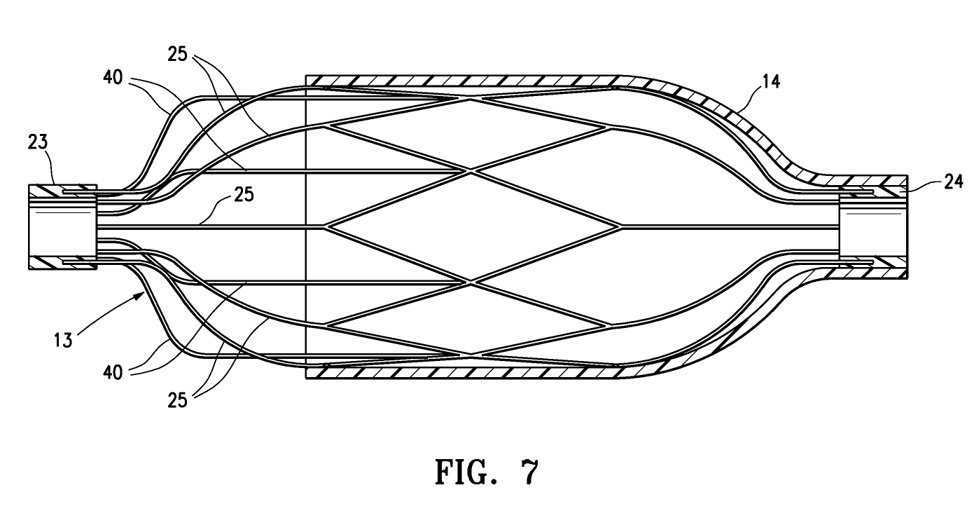

The sleeve 14 is fixedly secured to the struts, typically on an outer surface thereof, although the sleeve 14 can alternatively or additionally be secured to an inner surface of the frame 13. The open proximal end 30 of the sleeve forms a sleeve mouth located between the proximal and distal ends of the frame 13, such that the frame has a first longitudinal section along which the sleeve does not extend and a second longitudinal section along which the sleeve does extend. In the illustrated embodiment, the opposite end of the sleeve 14 is a closed end sealingly secured around the distal skirt section 24 of the frame. Thus, the sleeve is a blind sack preventing fluid flow through the sleeve outside of the skirt section 24. The sleeve mouth 30 has a uniform circular shape 14, with the sleeve 14 having a substantially uniform length around the circumference of the frame in the illustrated embodiment. FIG. 7 is a longitudinal cross sectional view of the covered frame 13 of FIG. 1, illustrating the frame 13 inside the sleeve 14, but with the catheter shaft 11 not shown for ease of illustration.

The frame 13 has sleeve-folding struts 40 which extends at least along at least a part of the first longitudinal section of the frame. In the illustrated embodiment, the sleeve-folding struts 40 extend along the entire length of the first longitudinal section of the frame and along part of the length of the second (i.e., sleeved) longitudinal section of the frame. Specifically, the sleeve-folding struts 40 extend from the proximal skirt section 23 to a location distal to the proximal end mouth 30 of the sleeve 14 and proximal to the distal skirt section 24. The sleeve-folding struts 40 each have a larger outer diameter in the expanded configuration than strut 25 portions circumferentially adjacent thereto, to radially collapse prior to the circumferentially adjacent strut 25 portions and thereby fold the sleeve 14 inwardly as the frame 13 radially collapses.

It should be understood that a variety of suitable frame configurations can be used in a device of the invention, with a different number or configuration of structural struts 25 than illustrated in the embodiment of FIG. 1. In the embodiment in which the sleeve is solid-walled and occludes the body lumen, as the frame initially begins to open, the sleeve begins to fill with blood/fluid flow in the body lumen such that the flow acts to force the sleeved frame further open until the frame seals against the inner surface of the body lumen wall. Thus, the sleeve supplements the radially expansive force of the frame, such that a frame design which provides a relatively small expansive force will nevertheless be caused to affectively seal within the body lumen. In one embodiment, the solid-walled sleeve is on a frame which has a relatively sparse connection of struts for improved flexibility, such that the frame has a radially self-expansive force insufficient to fully open the frame to its maximum radially expanded outer diameter, and after the initial radial self-expansion to a partially expanded configuration, the covered frame fully expands to its maximum diameter as the building pressure of blood in the interior of the covered frame trapped by the sleeve forces the frame to fully open. The frame 13 is typically formed by cutting the desired pattern into a wall of a tube, to form the expandable/collapsible network of struts. However, a variety of suitable methods can be used to form the frame including securing together a series of separate struts to form the frame. In the illustrated embodiment, the network of structural struts 25 have a diamond shaped pattern of cells which form the expandable/collapsible tubular body of the frame 13, and the distal end of each sleeve-folding strut 40 is fixedly secured at the apex of a diamond cell.

In an agent delivery procedure, the device 10 is advanced within the patient's body lumen with the outer sheath member 16 positioned around the frame 13 so that the frame is collapsed within the outer sheath member. Once at a desired location within the body lumen, the outer sheath member is proximally retracted to allow the frame 13 to radially self-expand to the expanded configuration illustrated in FIG. 1. With the frame radially expanded such that the sleeve contacts and seals against the inner surface of the wall of the patient's body lumen, an agent is delivered by infusing from an agent source (not shown) connected to the adapter port 19, through the infusion lumen 20 and out the agent delivery port 21. The agent can be delivered for a variety of treatment procedures, including treatment of a diseased (occluded) blood vessel by delivery of the agent directly into the diseased blood vessel, or treatment of the myocardium of the heart by delivery of an agent into one of the (healthy) coronary arteries. Additional interventional devices such as balloon angioplasty or stent delivery devices (not shown) can be used in conjunction with the device 10 during the treatment. After the initial infusion of agent, the device 10 may have to be repositioned, to complete the treatment of the site or to treat a different site. Significantly, the catheter 10 of the invention is configured to be readily collapsed as discussed in more detail below, which facilitates repositioning to allow for a complete, affective treatment, and/or removal from the patient when the treatment is finished.

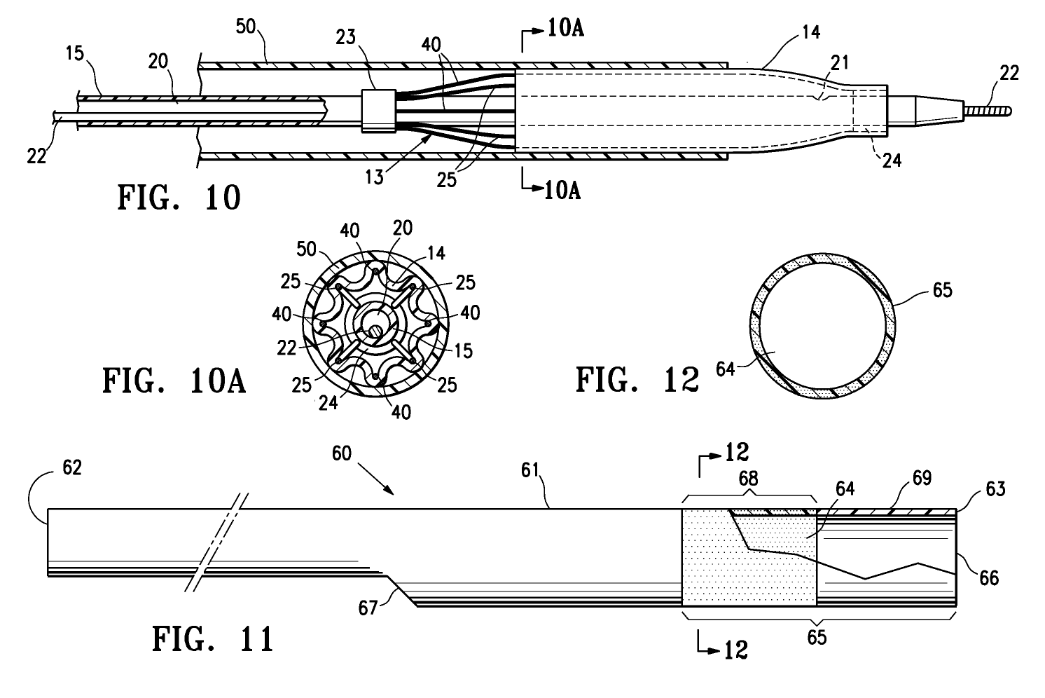

FIGS. 8-10 illustrate the collapse of the expanded frame 13 into a recovery catheter 50 to allow for the device 10 to be repositioned or removed from the patient's body lumen. Although the embodiment of the device illustrated in FIGS. 8-10 has four structural struts 25 and four sleeve-folding struts 40 along the proximal end section of the frame, a variety of suitable frame configurations can be used depending on factors such as the desired expansive force characteristics of the frame, as discussed above. The recovery catheter 50 is typically loaded onto the proximal end of the catheter 10 after any other interventional devices have been removed therefrom. Additionally, in the embodiment illustrated in FIG. 8 in which a separate recovery catheter 50 is used, the outer member (delivery sheath) 16 is typically removed from the inner tubular member 15, as discussed above, prior to advancement of the recovery catheter 50 over the inner tubular member 15. The recovery catheter 50 is slidably advanced over the shaft 11 (e.g., over the inner tubular member 15 following removal of the outer member 16) to position a distal end of the recovery catheter proximally adjacent to the radially expanded frame 13 (see FIG. 8). The expanded frame is then collapsed by slidably displacing the recovery catheter 50 relative to the first longitudinal section of the frame 13, such that the sleeve-folding struts 40 are contacted and radially collapsed by the recovery catheter prior to the struts 25 circumferentially adjacent thereto, to thereby fold the sleeve 14 inwardly as the frame 13 radially collapses. As illustrated in FIG. 9, as the distal end of the recovery catheter 50 is advanced distally along the conical proximal end of the frame 13, it first makes contact with the sleeve-folding struts 40 and begins to push the sleeve-folding struts inwardly before the circumferentially adjacent structural struts 25 begin to collapse. As a result, the sleeve 14 folds inwardly along each collapsing sleeve-folding strut secured thereto as best illustrated in FIG. 9A showing a transverse cross section of the device assembly of FIG. 9 taken along line 9A-9A. The distally advancing recovery catheter will eventually contact the structural struts 25 prior contacting the sleeve mouth 30 in the illustrated embodiment, such that the tubular body of the frame is caused to radially collapse by pivoting the struts down toward the inner member 15, as the sleeve-folding struts pull the sleeve 14 taught and inwardly towards the center of the frame 13. The collapsing sleeve 14 therefore will not bunch or fold outwardly along the collapsing frame 13, and the recovery catheter 50 can be advanced distally over the sleeved section of the collapsing frame 13, to fully the collapse the frame 13 (see FIGS. 10 and 10A) with the sleeve in a compact, low profile, configuration gathering generally inwardly through the struts of the frame 13. In the embodiment illustrated in FIG. 10, the frame 13 is fully collapsed within the recovery catheter 50, with the distal end of the recovery catheter 50 proximal to the distal end of the frame 13, although the recovery catheter 50 can alternatively be distally advanced further towards the distal end of the frame 13 prior to repositioning or removal of the device 10 from the body lumen.

After the infusion of the agent at the initial site, in order to extend the length of the treated site or treat a different diseased location, the catheter 10 having the frame in the collapsed configuration in the recovery catheter is repositioned and the frame re-expanded at the new location in the patient's body lumen, to allow for infusion of agent at the new location. Thus, the frame 13 of agent delivery catheter 10 may be repeatedly expanded and collapsed multiple times before finally being collapsed into recovery catheter 50 and removed from the patient.

The minimum number of sleeve-folding struts to act upon the sleeve (i.e., keep the sleeve 14 from gathering and bunching outside of the collapsing frame) is preferable in order to avoid disadvantageously increasing the stiffness of the distal end of the catheter 10. Typically, the frame has at least three sleeve-folding struts 40 for a sleeve 14 extending fully around the circumference of the frame. Preferably, a sleeve-folding strut 40 is provide between each adjacent pair of longitudinally extending structural struts 25 along the conical proximal end of the frame. Thus, in the embodiment illustrated in FIG. 1 in which the frame 13 has eight structural struts 25 along the conical proximal section of the frame, a total of eight sleeve-folding struts 40 are uniformly distributed around the circumference of the conical proximal end of the frame between each adjacent pair of structural struts 25. In one presently preferred embodiment, the frame has no more than about six structural struts 25, and more specifically has four structural struts 25 and four sleeve-folding struts 40 interspersed therebetween along the conical proximal section of the frame, so that the frame doesn't radially expand with a potentially harmful amount of force and/or isn't overly stiff. Although a presently preferred frame design has about three to about six sleeve-folding struts 40, it should be understood that a variety of suitable frame and sleeve designs could be used requiring more or less sleeve-folding struts.

The sleeve 14 preferably extends along more than half of the length of the frame 13, and the sleeve mouth 30 in the illustrated embodiments has a continuous circular shape (see FIG. 4B). The sleeve 14 is thus preferably configured to provide a relatively large area that facilitates expanding and sealing against the patient's vessel wall. Although the open end of the sleeve 14 in alternative embodiments (not shown) can follow along and correspond to the pattern of the structural struts 25, so that the mouth of the sleeve would have a zigzag shape in the illustrated embodiments, the continuous circular shape of the sleeve mouth 30 in the illustrated embodiment results in sections of the sleeve 14 at the mouth 30 which are extending between and not supported by the structural struts 25 of the frame 13 and which thus significantly benefit from the action of the sleeve-folding struts 40. The sleeve-folding struts 40 thus act to pull in the mouth 30 of the sleeve 14 as the recovery catheter 50 approaches, although they may act to additionally or alternatively pull in sections of the sleeve 14 distal to the mouth 30 which otherwise could bunch outwardly during collapse of the frame 13.

The sleeve-folding struts 40 preferably extend along a length of the sleeve 14, distal to the mouth 30, to directly apply a folding force to the sleeve therealong. Less preferred, due at least in part to issues relating to frame manufacturability, are sleeve-folding struts having a distal end at (not longitudinally spaced distally from) the mouth 30 of the sleeve. In the illustrated embodiment, the sleeve-folding struts 40 extend along about one third of the collapsing/expanding length of the sleeve 14, although more generally they may extend along about 25% to about 35% percent of the collapsing/expanding length of the sleeve (excluding the distal skirt section 24 length of the sleeve). The sleeve-folding struts 40 preferably do not extend the full length of the sleeve 14, for improved frame flexibility.

As illustrated, the frame 13 is preferably oriented to collapse from the proximal toward the distal end thereof into a recovery catheter advanced distally over the elongated shaft of the catheter 10. However, the frame could alternatively be flipped to orient it for collapsing from the distal toward to the proximal end of the frame, typically by providing the catheter with a distal tip recovery sleeve configured for being remotely retracted proximally to collapse the frame therein, typically for use in larger peripheral vessels. Thus, although the sleeve is illustrated with an open proximal end and a closed distal end, it should be understood that the sleeve on the frame generally has an open first end forming a sleeve mouth located between the proximal and distal ends of the frame and an opposite end, which in one embodiment (not shown) is a open distal end and closed proximal end. Similarly, it should be understood that the sleeve-folding struts 40 can be used with a variety of covered frame devices having one or more frames to facilitate recovery of the device, with the sleeve-folding struts extending from one or more open mouths of the frame cover. For example, in one embodiment (not shown), both ends of the sleeve are open like mouth 30 such that the sleeve defines an open passageway therethrough.

FIG. 11 illustrates a porous recovery catheter 60 found useful in a catheter system embodying features of the invention, and FIG. 12 illustrates a transverse cross section of the catheter 60 taken along line 12-12. The porous recovery catheter 60 generally comprises a shaft 61 having a proximal end 62, a distal end 63, a single lumen 64 extending at least in a distal shaft section, and a porous wall along at least a portion of a distal recovery section 65. The distal recovery section 65 is the section configured to slidably receive the collapsing frame 14 of device 10 therein.

The porous recovery catheter 60 is configured for recovery of an expandable frame device such as catheter 10. Thus, the recovery catheter 60 has a distal port 66 configured to allow the distal recovery section to be slidably advanced over the expanded frame 13 to thereby radially collapse the frame 13 from the expanded to the collapsed configuration, and allow fluid flow through the porous wall of the recovery section 65. The porous region of the distal recovery section 65 has a porosity configured to allow fluid forced by pressurization through the porous wall as the frame is collapsed into the recovery section of the shaft, wherein the porosity is sufficiently small such that the porous wall has sufficient column strength for collapsing the frame. In one embodiment, the porous wall comprises a plurality of pressure relief ports with pore sizes which are about 150 to about 200 micrometers (μm). Preferably the pore size is sufficient for a quick and low pressure release of the trapped fluid, and the fluid flows out of the recovery catheter through the pores once the pressure of the fluid is slightly above the blood pressure of the vessel.

In the illustrated embodiment, the recovery catheter 60 is a rapid-exchange type catheter such that the lumen 64 extends from the distal tip of the catheter 60 to a proximal rapid-exchange port 67 at a location distally spaced from the proximal end 62 of the recovery catheter 60. The proximal section of the catheter shaft 61 (i.e., proximal to the rapid exchange port 67) is typically a tubular member, although with a smaller lumen size than along the distal recovery section 65 of the catheter 60. The port 67 is configured to allow the shaft 11 of catheter 10 to slidably extend therethrough. Alternatively, port 67 can be omitted such that the entire length of the recovery catheter 60 is slidably advanced over the shaft 11 of device 10. For use as a recovery catheter, the lumen 64 has its largest diameter from the distal most end of the catheter 60 at port 66 and extending proximally therefrom along the distal recovery section 65 of the shaft, in order to be slid over the frame 14 to collapse the frame 14. Thus, the lumen 64 does not taper to a smaller inner diameter along the distal recovery section 65 (i.e., the lumen inner diameter does not decrease from the porous portion to the distal-most end of the recovery catheter).

In the illustrated embodiment, only a portion 68 of the distal recovery section 65 is porous and the shaft 61 has a soft distal tip member 69 secured to the distal end of the porous section. The relatively flexible polymeric tip member 69 facilitates atraumatic advancement of the catheter 60 within the patient's body lumen and provides for a smoother recovery (i.e., ease of advancement of the catheter 60 over a covered frame of a device such as catheter 10). In a preferred embodiment, the distal tip member 69 is solid-walled (i.e., non-porous), such that the porous region is preferably limited to the portion of the recovery catheter that will be at the mouth of the collapsing sleeve 30, and specifically where the mouth 30 of the collapsed sleeve 14 will come to rest in the recovery catheter 60. Minimizing the length of the porous region provides an exit path for blood and contrast where it is needed, while also providing improved stability at the distal tip. In an alternative embodiment, the porous wall extends along the distal tip to the distal-most end of the recovery catheter. Thus, pores are typically only needed at the proximal end of the tip, although pores could be needed along the entire tip depending on factors such as the configuration of the device to be recovered. The distal tip can be a separate member bonded to the proximally adjacent section of the shaft, or alternatively an integral one-piece extension of the wall forming the porous section.

By allowing fluid (e.g., blood and contrast) in the collapsing frame 13 to exit through the porous region 68, recovery of the frame 13 is facilitated. Although the pressure build-up caused by trapped fluid is greatest with a frame covered by a solid-walled sleeve designed for occluding the patient's blood vessel, a sleeve which limits but doesn't eliminate all blood flow through the sleeve still benefits from the porous recovery catheter.

Although discussed primarily for use in a catheter system with the catheter 10, it should be understood that the porous recovery catheter 60 embodying features of the invention can be used to recover a variety of suitable devices. The porous recovery catheter typically has a length of about 150 cm to about 180 cm. In one embodiment, the distal recovery section 65 has a length of about 1.0 to about 3.0 cm, more typically about 2.0 cm, an outer diameter of about 0.15 to about 0.20 cm, and an inner diameter of about 0.1 to about 0.14 cm, and the porous portion 68 has a length of about 0.5 to about 1.0 cm, more typically about 0.75 cm.

Although the catheter 10 is discussed primarily in terms of an embodiment in which the catheter 10 is configured for agent delivery and has a solid-walled occluding sleeve on the frame, it should be understood that the frame, which in accordance with the invention has at least one sleeve-folding strut 40, can be used on a variety of suitable devices, including an embolic protection device. In an embolic protection device not configured for agent delivery, the frame 13 typically has a permeable filtering sleeve configured to allow the flow of fluid (blood) through the wall of the sleeve in the expanded configuration, and the frame is typically directly mounted to core wire 22 without the agent delivery lumen 20. Thus, it should be understood that the shaft of a device of the invention, onto which the sleeved frame is mounted, can be a lumen-defining tubular member, or only a core wire.

The dimensions of catheter 10 depend upon factors such as the catheter type and the size of the artery or other body lumen through which the catheter must pass. By way of example, the outer sheath member 16 typically has an outer diameter of about 0.025 to about 0.04 inch (0.064 to 0.10 cm), usually about 0.037 inch (0.094 cm), and a wall thickness of about 0.002 to about 0.008 inch (0.0051 to 0.02 cm), typically about 0.003 to 0.005 inch (0.0076 to 0.013 cm). The inner tubular member 15 typically has an inner diameter of about 0.01 to about 0.018 inch (0.025 to 0.046 cm), usually about 0.016 inch (0.04 cm), and a wall thickness of about 0.002 to about 0.004 inch (0.005 to 0.01 cm). The overall length of the catheter 10 may range from about 100 to about 150 cm, and is typically about 143 cm. Typically, for coronary arteries, frame 13 has a length about 0.8 cm to about 6 cm, and a radially expanded outer diameter of about 2 to about 5 mm.

A variety of suitable agents can be delivered using the catheter(s) and method(s) of the invention, including therapeutic and diagnostic agents. The agents are typically intended for treatment and/or diagnosis of coronary, neurovascular, and/or other vascular disease, and may be useful as a primary treatment of the diseased vessel, or alternatively, as a secondary treatment in conjunction with other interventional therapies such as angioplasty or stent delivery. Suitable therapeutic agents include, but are not limited to, thrombolytic drugs, anti-inflammatory drugs, anti-proliferative drugs, drugs restoring and/or preserving endothelial function, and the like. A variety of bioactive agents can be used including but not limited to peptides, proteins, oligonucleotides, cells, and the like. A variety of diagnostic agents can be used according to the present invention. According to the present invention, agents described herein may be provided in a variety of suitable formulations and carriers including liposomes, polymerosomes, nanoparticles, microparticles, lipid/polymer micelles, and complexes of agents with lipid and/or polymers, and the like.

In a presently preferred embodiment, catheter 10 of the invention is configured for delivery of an agent to a coronary artery or vein, for example for the treatment of a diseased/occluded region of the artery or vein or for the treatment of the adjacent myocardium of the heart wall. However, the vasculature need not be coronary, and can be, for example, renal, femoral, popliteal, carotid, cerebral or other arteries and veins, aneurysms and aneurismal sacs, and may include delivery to implanted devices therein such as grafts, stents and the like. Similarly, agent delivery may occur to the wall of a variety of tubular body lumens including pulmonary, gastrointestinal and urinary tract structures. Thus, the term “vessel” as used herein should be understood to refer generally to body lumens.

Although discussed primarily in terms of a preferred self-expanding frame 13 on catheter 10, the frame could alternatively be configured to radially expand upon operation of an activation member forcing the frame open. However, a self-expanding frame is preferred, at least in part to provide for easy repositioning (collapse and redeployment), and to provide the catheter of the invention with a relatively low profile and high flexibility, which facilitates positioning the operative distal end of the catheter within the vasculature.

The frame 13 is typically formed of a metal such as stainless steel or a NiTi alloy. A variety of suitable materials can be used to form the sleeve 14 including polyurethane, a polyether block amide (PEBAX), and nylon, which can be formed into films, membranes, or woven structures to form the sleeve 14. In a presently preferred embodiment, the sleeve is formed of a polyurethane polymeric material. The sleeve 14 is bonded to an outer surface of the frame 13 with heat bonding, although an adhesive could additionally or alternatively be used. In one embodiment, the heat bonding melts the sleeve, causing it to flow around the struts of the frame and bond to itself, thus encapsulating the struts. The shaft tubular members can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives.

While the present invention is described herein in terms of certain preferred embodiments, those skilled in the art will recognize that various modifications and improvements may be made to the invention without departing from the scope thereof. For example, the catheters can be designed to have multiple frames (e.g., a bifurcated catheter), and a dilatation/stent delivery balloon can be added to the catheter proximal or distal to the frame to allow the catheter to perform the dual functions of agent delivery and balloon angioplasty/stent delivery. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

We claim:

1. A device having an expandable frame configured for reversibly expanding in a patient's body lumen, comprising:
a) an elongated shaft having a distal shaft section; and
b) an expandable frame on the distal shaft section, configured to radially collapse from an expanded configuration as a recovery catheter is slidably advanced over the expanded frame, having a plurality of struts, a proximal end, a distal end, and a sleeve fixedly secured to the struts with an open first end forming a sleeve mouth located between the proximal and distal ends of the frame and an opposite end such that the frame has a first longitudinal section along which the sleeve does not extend and a second longitudinal section along which the sleeve does extend, and having at least one sleeve-folding strut which extends at least along at least a part of the first longitudinal section of the frame, and which has a larger outer diameter in the expanded configuration than strut portions circumferentially adjacent thereto, to radially collapse prior to the circumferentially adjacent strut portions and thereby fold the sleeve inwardly as the frame radially collapses.

2. The device of claim 1 wherein the frame has at least three sleeve-folding struts circumferentially spaced around a circumference of the frame.

3. The device of claim 1 wherein the frame has a proximal skirt section and a distal skirt section mounting the frame on the distal shaft section, with the struts extending from the proximal to the distal skirt section of the frame.

4. The device of claim 3 wherein the sleeve-folding strut has a proximal end at the proximal skirt section of the frame.

5. The device of claim 1 wherein the open end of the sleeve is the sleeve proximal end, and the opposite end of the sleeve is the sleeve distal end, and the sleeve-folding strut has a distal end at a location distal to the open proximal end of the sleeve and proximally spaced from the distal end of the sleeve.

6. The device of claim 1 wherein the sleeve extends along more than half of the length of the frame, with the sleeve mouth having a continuous circular shape.

7. The device of claim 1 wherein the sleeve is a solid-walled occluding sleeve configured to prevent the flow of fluid through the sleeve wall in the expanded configuration.

8. The device of claim 1 wherein the sleeve is a permeable filtering sleeve configured to allow the flow of fluid through the sleeve wall in the expanded configuration.

9. The device of claim 1 wherein the device is an agent delivery catheter, and the elongated shaft has at least one lumen which is configured for fluid delivery of an agent and which extends from a proximal end of the catheter to an agent delivery port located in the distal shaft section.

10. The device of claim 9 wherein the agent delivery port is distal to a distal end of an interior of the sleeve.

11. The device of claim 1 wherein the opposite end of the sleeve is a closed end secured around the shaft.

12. An agent delivery catheter having a self-expanding frame configured for reversibly expanding in a patient's body lumen, comprising:
a) an elongated shaft having a distal shaft section, and an agent delivery lumen extending therein to an agent delivery port in the distal shaft section; and
b) a self-expanding frame on the distal shaft section, configured to radially collapse from an expanded configuration as a recovery catheter is slidably advanced over the expanded frame, having a plurality of struts, a proximal end, a distal end, and a solid-walled sleeve fixedly secured to the struts with an open proximal end forming a sleeve mouth located between the proximal and distal ends of the frame and a distal end, such that the frame has a first longitudinal section along which the sleeve does not extend and a second longitudinal section along which the sleeve does extend, and having a least one sleeve-folding strut which extends at least along at least a part of the first longitudinal section of the frame, and which has a larger outer diameter in the expanded configuration than strut portions circumferentially adjacent thereto, to radially collapse prior to the circumferentially adjacent strut portions and thereby fold the sleeve inwardly as the frame radially collapses.

13. The agent delivery catheter of claim 12 wherein the sleeve extends along more than half the length of the self-expanding frame, with the sleeve mouth having a continuous circular shape.

14. A catheter system configured for reversibly deploying in a patient's body lumen, comprising:
a) a device comprising an elongated shaft, and a self-expanding frame on a distal shaft section, the frame being configured to reversibly transform from a collapsed configuration to an expanded configuration, and the frame has a plurality of struts, a proximal end, a distal end, and a sleeve fixedly secured to the struts with an open proximal end located between the proximal and distal ends of the frame and a distal end, such that the frame has a first longitudinal section along which the sleeve does not extend and a second longitudinal section along which the sleeve does extend, and having a least one sleeve-folding strut which extends at least along at least a part of the first longitudinal section of the frame and which has a larger outer diameter in the expanded configuration than strut portions circumferentially adjacent thereto, to radially collapse prior to the circumferentially adjacent strut portions and thereby fold the sleeve inwardly as the frame radially collapses; and
b) a recovery catheter slidably disposed on the device elongated shaft, having a distal recovery section with a porous wall configured to be slidably advanced over the expanded frame to thereby radially collapse the frame from the expanded to the collapsed configuration and allow fluid flow through the porous wall.

15. A recovery catheter, comprising an elongated shaft which has a single lumen extending to a distal port at an open distal-most end of the recovery catheter and dimensioned for slidably advancing over a device having a reversibly expandable frame to thereby collapse the expanded frame to a collapsed configuration, and which has distal recovery section, and a porous wall along at least a portion of the distal recovery section of the shaft with a porosity configured to allow fluid forced by pressurization through the porous wall as the frame is collapsed into the recovery section of the shaft, wherein the porosity is sufficiently small such that the porous wall has sufficient column strength for collapsing the frame, and the lumen has an inner diameter that does not decrease from the porous portion to the distal-most end of the recovery catheter.

16. The recovery catheter of claim 15 wherein the porous wall comprises a plurality of pressure relief ports with pore sizes which are about 150 to about 200 micrometers.

17. The recovery catheter of claim 15 wherein the lumen extends from the distal port to a proximal rapid-exchange port located distally spaced from the proximal end of the recovery catheter.

18. A method of recovering a device having an elongated shaft and an expanded frame on a distal shaft section configured for reversibly transforming from a radially expanded configuration to a collapsed configuration in a patient's body lumen, comprising:
a) slidably advancing a recovery catheter over the device to position an end of the recovery catheter adjacent to the radially expanded frame, the frame having a plurality of struts, a proximal end, a distal end, and a sleeve fixedly secured to the struts with an open first end located between the proximal and distal ends of the frame and an opposite end, such that the frame has a first longitudinal section along which the sleeve does not extend and a second longitudinal section along which the sleeve does extend, and having a least one sleeve-folding strut which extends at least along at least a part of the first longitudinal section of the frame, and which has a larger outer diameter in the expanded configuration than strut portions circumferentially adjacent thereto; and
b) collapsing the frame by slidably disposing the first longitudinal section of the frame within the recovery catheter, such that the sleeve-folding strut contacts and is radially collapsed by the recovery catheter prior to the circumferentially adjacent strut portions, to thereby fold the sleeve inwardly as the frame radially collapses.

19. The method of claim 18 wherein the sleeve-folding strut and the circumferentially adjacent struts have equal outer diameters along a portion thereof, and collapsing the frame includes further advancing the recovery catheter along the first longitudinal section of the frame to the equal diameter portions of the struts, to contact said circumferentially adjacent struts along with the sleeve-folding strut and thereby further radially collapse the frame.

20. The method of claim 18 including further advancing the recovery catheter to the second longitudinal portion of the frame having the sleeve, and wherein the recovery catheter comprises an elongated shaft having a porous wall along at least a portion of a distal recovery section of the shaft with a porosity such that advancing the recovery catheter over the second longitudinal portion of the frame collapses the frame and thereby forces fluid from within the sleeve through the pores to outside of the recovery catheter, wherein the porosity is sufficiently small such that the porous wall has sufficient column strength for collapsing the frame.
